(19) United States
(12) Patent Application Publication
(10) Pub. No.: US 2022/0341929 A1
(43) Pub. Date: Oct. 27, 2022

(54) Point of Sampling Kit and Method for Assessing Endotoxin Contamination

(71) Applicants: Thomas J Novitsky, East Falmouth, MA (US); Erik J Paus, Brewster, MA (US)

(72) Inventors: Thomas J Novitsky, East Falmouth, MA (US); Erik J Paus, Brewster, MA (US)

(21) Appl. No.: 17/660,039

(22) Filed: Apr. 21, 2022

Related U.S. Application Data
(60) Provisional application No. 63/180,225, filed on Apr. 27, 2021.

(51) Int. Cl. G01N 33/569; G01N 33/579

(52) U.S. Cl. CPC G01N 33/56911; G01N 33/579; G01N 2400/50

(57) ABSTRACT

A point-of-use kit is designed for optically detecting and quantifying bacterial endotoxin by employing specific formulations of Limulus amebocyte lysate (LAL), each formulation designed to optimize results with different sample classes. Kits are pre-certified for use with a variety of environmental, industrial, and clinical samples, each sample category having a unique kit design and containing a unique lysate reagent formulation. Pre-certification transfers time and reagent consuming tasks, such as assessment of sample compatibility and sample effect on reagent sensitivity to endotoxin from the user to the kit producer. A fixed dilution/sample treatment is employed, eliminating the need for a comparative water standard and for a sample positive control. The kit has LAL reagent prepackaged in dry polyethylene capped tubes, which retains reagent shelf life and optical clarity for accurate and reproducible results using a portable spectrophotometer/optical reader.

### RELATED APPLICATION

This application claims the benefit of provisional Pat. App. Ser. No. 63/180,225, filed on Apr. 27, 2021.

### FIELD OF THE INVENTION

This invention relates to point-of-use kits employing unique formulations of Limulus amebocyte lysate (LAL) designed to be compatible with various industrial, clinical, and environmental samples that eliminate excessive water dilution in order to eliminate sample interference found with currently employed LAL reagents, methods, and kits.

### BACKGROUND OF THE INVENTION

The Limulus amebocyte lysate (LAL) assay, soon after its discovery in the late 1960's, rapidly became the method of choice by researchers interested in the detection and quantitation of endotoxin in a variety of samples. Initially, and perhaps due to the interest of the physician who discovered the assay, most research focused on the detection of endotoxin in blood. This application was based on the fact that circulating endotoxin in blood could be an indicator of Gram-negative bacteremia and bacterial sepsis. The more general finding that LAL-detected endotoxin was related to bacterial infection soon led to the use of LAL to study urinary tract infections (UTI's), spinal meningitis, and even gonorrhea. LAL use then expanded to study endotoxin in air due to particulates generated from agricultural and industrial processes, e.g., cotton milling, metal cutting, animal husbandry, cooling towers, and internal engine combustion exhaust. Airborne endotoxin in all these situations collected by impingement and put into an aqueous solution for testing with LAL was found to correlate with various respiratory ailments and other diseases. Other environmental applications included the examination of water and wastewater, food and beverage production, assessing dairy product shelf life, assessing fish spoilage, and more personal applications including measuring endotoxin in steam (steam baths), assessing the cleanliness of personal breathing apparatus, and measuring endotoxin in exhaled smoke from cigarettes. LAL was also found to be useful in assessing contamination of solutions used in renal dialysis and to determine the efficacy of cleaning/sanitizing dialysis equipment. In all cases, the LAL assay was shown to be the preferred, if not the only method for the rapid and cost-effective detection and quantitation of endotoxin.

While LAL became the method of choice for endotoxin detection, the assay remained a laboratory, rather than a point-of-use (POU) or point-of-sampling (POS) test, with its most widespread use dedicated to controlling endotoxin (a pyrogenic or fever causing) bacteria-related contamination potentially arising during the preparation of injectable drugs and medical devices. Thus, LAL became a laboratory test for pharmaceutical quality control/assurance and an official (pyrogen) test required for final product release for human and animal injectable products.

The reason(s) the LAL test did not gain acceptance as a POU/POS test are many and varied but basically the assay was designed and commercialized specifically for testing solutions, devices, and raw materials associated with pharmaceutical drug production. This specific laboratory application along with very specific testing regimes, governmental agency approved methods and reagents, and kit designs is the primary application for LAL today. This use is highly regulated by various governmental agencies and non-governmental organizations. As a result, all commercially available kits currently available follow the same basic protocol and are designed to measure endotoxin compared to a water standard.

The most common ingredient in the preparation of injectable drugs is purified water. Purified water that contains very little or no endotoxin is also used to prepare LAL, reconstitute freeze-dried LAL reagent, prepare endotoxin standards, and also dilute drug samples in preparation for LAL testing. It was quickly found that most drug formulations, i.e., water containing various salts and/or other chemicals, inhibited the LAL reagent to some degree. This inhibition was commonly overcome by diluting the drug with pure water, i.e., water containing little to no endotoxin. This dilution effectively made the sample more like pure water by lowering the concentration of inhibitory substances. The obvious drawbacks to dilution were not only increased assay time but also a lowering of the endotoxin concentration able to be detected, i.e., assay sensitivity. For example, an LAL reagent shown to detect a minimum of 1 Endotoxin Unit (EU) in a water sample, would detect less if the sample were diluted with additional pure water, e.g., a sample requiring 1:10 dilution would now contain only 0.1 EU's representing 10 EU's in the original, i.e., undiluted sample. The United States Pharmacopeia (USP) and other pharmacopeias worldwide publish standardized methods for testing drugs with LAL, providing specific procedures for determining if and how much dilution is required for a valid LAL test. Since dilution effectively lowers the detection limit of LAL, many drugs requiring dilution to overcome interference with the LAL reagent must use a higher sensitivity LAL in order to assure the level of endotoxin in their drugs meet statutory requirements. Standardized methods also include a standard endotoxin series using a certified endotoxin standard diluted in water, a test indicating that sample inhibition has been overcome when standard endotoxin is added to the product or product dilution, and a positive product (sample) control. As environmental, clinical, and industrial samples often contain levels of endotoxin well above those found in the pharmaceutical industry, these types of controls are of doubtful usefulness. This invention eliminates the need for these controls.

Likewise, clinical samples such as blood, serum and plasma, also contain interfering factors that must be removed via dilution in pure water and in some cases, using additional procedures, such as heating for certain times/temperatures.

It has been shown that most samples that interfere with LAL do so in a stoichiometric manner, i.e., the interference becomes less with additional dilution in water. Thus, if there were a way to quantitate the reaction of LAL in a sample without complete or near complete removal of sample interference, an advantage could be gained, saving time and eliminating chance of error.

### SUMMARY OF THE INVENTION

It is the object of the present invention to provide a kit design for the simple and rapid measurement of endotoxin in a variety of sample types.

The present invention provides a reagent and accompanying kit that has been designed to eliminate excess dilution by assigning a sensitivity to LAL at a predetermined, single dilution, of specific sample types. This attribute and other design criteria of the invention also allow assays to be conducted outside a testing laboratory, i.e., at the point where samples are collected.

The current invention also provides a means to use the LAL assay contained in kits designed specifically for assessing endotoxin contamination in a variety of clinical, industrial and environmental applications (i.e., this kit is not intended for regulated pharmaceutical use). Within the pharmaceutical industry, endotoxin contamination is rare due to a myriad of controls generally known as Good Manufacturing Practices. Outside the pharmaceutical industry however, endotoxin contamination is extremely common, although for the most part, endotoxin, unless injected into the bloodstream, associated with Gram-negative bacterial infection, or inhaled in large concentrations, is basically not a health concern. For example, in addition to all the non-pharmaceutical applications listed above, one particularly important kit designation will be for renal and peritoneal dialysis contamination control, at the Point-of-Sampling (POS). (Note: the term Point-of-Sampling (POS) will be used here but should be considered synonymous with the terms Point-of-Care (POC) and Point-of-Use (POU). Other potential applications include: contamination control of devices used for sleep disorders; assessing household and industrial mold contamination (requires an additional reagent in the kit); assessing adequacy of home water purification systems; and many other clinical, industrial, agricultural or worker/consumer safety applications where the recognition and amount of microbial, i.e., bacterial contamination would be of benefit. All of these applications are associated with high concentrations of endotoxin when compared to the low or non-detectable levels found in pharmaceuticals.

As a POS test kit, this invention lends itself to using any number of commercially available portable, battery-powered, handheld spectrophotometers, or a purpose-built optical reader in order to accurately quantify results that can read turbidity as optical density (O.D.).

The novel features which are considered as characteristic of the invention are set forth in particular in the appended claims. The invention, itself, however, both as to its design, construction and use, together with additional features and advantages thereof, are best understood upon review of the following detailed description with reference to the accompanying drawings.

### BRIEF DESCRIPTION OF THE DRAWINGS

FIG. 1 shows the components of the testing kit and additional testing components of the present invention.

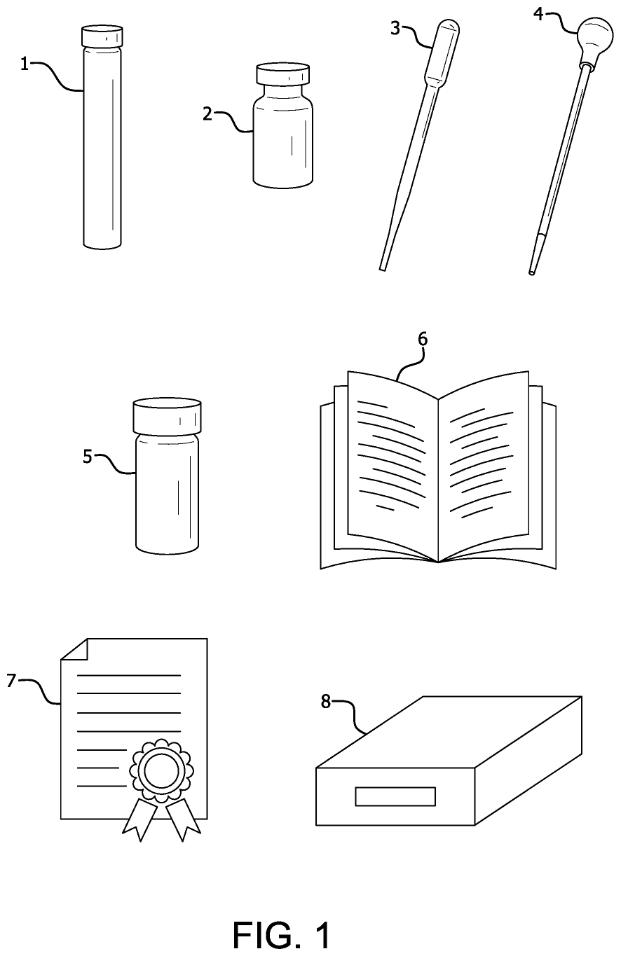

FIG. 2 is a flow diagram illustrating the steps of a representative method of the present invention, detecting and measuring bacterial endotoxins in an aqueous solution using optical measurement. The diagram is directed to the use of the testing kit for bovine serum.

### DETAILED DESCRIPTION OF THE INVENTION

In accordance with the invention, there is a process for preparing LAL (formulation) to increase its sensitivity to endotoxin by neutralizing or partially neutralizing sample inhibition. This process allows for assigning a sensitivity of the LAL as long as endotoxin can be reproducibly measured in a specific sample type. This is in contrast to the traditional method of diluting a sample in water until there is no detectable inhibition as observed with the addition of a known amount of endotoxin to the sample, i.e., a positive product control.

There are minimal criteria than can be assigned to LAL sensitivity. As a basis for this invention, the starting LAL formulation should possess (a) the ability to form sufficient turbidity to be detected in a spectrophotometer or optical reader utilizing a wavelength between 405 and 650 nm; (b) the maximum optical density achievable of at least 1.0 O.D. unit; (c) the ability to achieve the aforementioned criteria when a total of 1.0 mL of sample (containing LAL) is contained in a 12 mm diameter test tube; (d) the ability to achieve the aforementioned criteria when the test tube is either polystyrene or glass, have no detectable endotoxin when assayed with water that has been certified to contain no detectable endotoxin and are sterile; (e) the ability to achieve the aforementioned criteria when LAL can be lyophilized (freeze-dried) in the tube such so that it can be rehydrated with 1.0 mL of sample to initiate the assay; (f) the ability to have additional components added or changed to the basic formulation that may increase but not significantly lower the LAL sensitivity. Multiple lots have been produced using the above criteria and have been shown to have shelf lives in excess of 28 weeks when stored at either −20° C. or +2-8° C. In addition, reproducibility with a standard endotoxin concentration measured with the Serum Test Kit protocol (Example I), showed less than 0.7% variance (n=21).

Such LAL minimal criteria can be achieved using amebocytes collected from any of the four (4) extant species of horseshoe crabs, i.e., Limulus polyphemus (North America); Tachypleus tridentatus, Tachypleus giga, and Carcinoscopius rotundacauda (Asia). LAL from Limulus polyphemus being preferred.

### Collection of Limulus Amebocytes

Limulus Amebocytes were collected using the following modification of a basic procedure for producing LAL: Male and female North American horseshoe crabs, Limulus polyphemus, were collected near Assateague Island, Md. by trawling. Hemolymph (approximately 100 mL) was removed via cardiac puncture with a siliconized stainless steel 12-gauge needle after cleaning the arthrodial membrane (anatomical structure situated midway along the hinge connecting the prosoma and opishosoma, i.e., just dorsal and external to the horseshoe crab's tube heart) with 70% isopropyl alcohol was collected into a sterile, endotoxin-free, 250 mL centrifuge bottle containing 100 mL of 3% NaCl and 2 mM caffeine, and 10 mM Tris HCl buffer, pH 7.4 prewarmed to 37° C. When full, bottles are balanced and centrifuged at 200×g for ten (10) minutes at room temperature to sediment the amebocytes. The supernatant (cell-free hemolymph) is then carefully decanted. Note: decanting of the hemolymph and all subsequent handling procedures where solutions are being transferred are conducted in a biological cabinet (Class II Type A2) to prevent contamination. The resulting pellet of amebocytes are resuspended in 20 mL of 3% NaCl solution w/o caffeine, transferred to a 50 mL conical dry heat sterilized/depyrogenated test tube. Alternatively, the suspension is transferred to a 50 mL polystyrene capped centrifuge tube certified to be sterile and pyrogen-free. After the initial transfer of 20 mL, an additional 20 mL of 3% NaCl solution is added to the original tube to collect any remaining cells then removed and added to the tube containing the first 20 mL transfer. This tube is either covered with Parafilm® if a glass tube is used, or capped if a plastic tube is used. When sufficient tubes have been collected, they are balanced and transferred to a centrifuge where they are spun at 200×g for 10 minutes at 2-8° C. Following centrifugation, the supernatant (cell-free wash solution) is discarded and the process repeated using fresh 3% wash solution and a fresh centrifuge tube. After discarding the second wash the amebocyte pellet is frozen in situ at −20-−80° C.

### Amebocyte Lysis, Basic Formulation, and Lyophilization of LAL

The following steps were performed in a biological cabinet: Thaw the number of tubes required for the size of lot to be produced. Aseptically remove cells from centrifuge tubes with a depyrogenated spatula. Weigh amebocytes in a sterile heat depyrogenated beaker. If cells have been frozen, keep at 4 C, until completely thawed. Add sterile, WFI to cells at a 6:1 ratio mL of WFI:gram of cells. Depyrogenate the equipment (probe and homogenizer) used to rupture the cells and solubilize the membrane and intracellular proteins. Cells are thoroughly homogenized in 20 second bursts on ice until a smooth and consistent slurry is generated and no cell clumps are visible. Transfer the lysed cells to an appropriately sized Erlenmeyer flask that has been dry heat depyrogenated. Cell slurry should not exceed 25% of the total volume of the flask. i.e.: 50 mL for 200 mL flask, 125 mL of slurry in a 500 mL. flask. Cover the flask with Parafilm to maintain sterility. The flask may now be removed from the sterile hood for additional downstream processing.

Place flask containing lysed cell slurry on an orbital shaker at 4 C. Secure flask on the surface of the shaker to ensure flask is not dislodged during shaking causing spilling or breakage. Adjust speed of shaker to insure adequate mixing of cells in WFI. Shake for 24 hours. Remove flask from shaker and transfer to sterile hood.

Carefully pipette cell slurry into sterile, depyrogenated centrifuge tubes. Each tube must contain equal weights of slurry to avoid imbalance of centrifuge. Weights of each tube should within 0.5 gm. Seal each centrifuge tube and transfer to pre-chilled centrifuge (4 C). Set spin for 20 mins at 1150×g. After end of spin cycle, remove tubes to sterile hood. Pour off lysate into endotoxin free vessel, carefully so as not to dislodge the pelleted cell debris. Store at 4 C. Final basic formulation of the LAL prior to lyophilization comprises the following: addition of raw lysate, i.e., cell-free aqueous extract of collected amebocytes as previously described (mechanical disruption followed by centrifugation preferred); 25-150 mM Trizma HCl (50 mM preferred), pH 7.2-7.6 (7.4 preferred); 0.1-0.5 M CaCl₂) (0.2 M preferred); 0.1-0.5 M MgSO₄ (0.2 M preferred). Additional components (enhancing agents) are added or basic components changed depending on the intended sample to be analyzed.

Following formulation, liquid LAL is dispensed into either glass or plastic 12×75 mm tubes (plastic preferred) shown to contain an undetectable level of endotoxin using a standard chromogenic LAL assay with a sensitivity of 0.001 EU/mL. Liquid LAL is added via pipette or other dispensing device at a volume from 0.1 mL to 1.0 mL (0.2 mL preferred) in a biological cabinet or class 100 clean room. Immediately following filling vials are partially capped to allow air exchange and transferred to a −20 to −80° C. freezer (−30 preferred) or a lyophilizer with a shelf temperature of at least −30° C. for storage until initiation of the lyophilization cycle under conditions to prevent contamination with endotoxin. A minimum of 24 hrs at a temperature of at least −30° C. is required to affect full freezing when the LAL is formulated with the maximum concentration of ingredients that lower the freezing point.

Once frozen, the product if not frozen in the lyophilizer (preferred) is transferred to the lyophilizer whose shelves have been prechilled to at least −30° C., and the lyophilization cycle begun. The cycle will vary depending on the predetermined eutectic point. The cycle is run until the product is considered dry using any standard technique, e.g., a thermistor probe inserted in one or more vials selected to represent the lyophilizer run. When the cycle is complete, the chamber is backfilled with dry nitrogen (preferred) or other dry, inert gas, e.g., argon. Backfilling is followed by mechanically fully inserting the stoppers. Alternately, stoppers can be inserted when the chamber is still under full vacuum, i.e., without backfilling.

Following full stopper insertion, vials are removed from the lyophilizer stored at −20° C. until final packaging and kitting. Following these conditions, the LAL product has been shown to be stable at temperatures between for a period of at least 28 weeks. Further studies have shown that LAL stored at 2-8° C. immediately following lyophilization are also stable for at least 28 weeks.

### Preparation of Standard Endotoxin Solutions

Endotoxin standards used assess sensitivity and stability of the LAL and to determine the optimal LAL formulation and dilution and, if required, extraction procedure for each sample type subject to this invention was a preparation of Sigma Etoxate® Endotoxin Standard, lot no. SLBB8062V certified to contain 10,900 EU/vial when compared to the USP Endotoxin Standard. To use, the Etoxate® vial was reconstituted with Water for Injection, USP Sterile Grade to a final concentration of 10,000 EU/mL. This solution was stored at 2-8° C. for up to 1 year and was used to make monthly working solutions of 100 EU/mL. Daily working solutions of 10 EU/mL concentration or less were used for all sensitivity, stability, or product evaluation and calibration and were discarded after 24 hr.

### Preparation of Extraction Solutions

Extraction solutions are needed for certain kits, e.g., those designed to test serum of plasma. These solutions can be a simple as Water for Injection, USP Sterile Grade, or solutions made with Water for Injection, USP Sterile Grade, to which various salts or other ingredients have been added. All extraction solutions are contained in 15 mL capacity high density polyethylene (HDPE) vials with a tightly sealing polypropylene cap. Vials are sterile and shown to contain less than 0.001 EU/mL of endotoxin when measured with a chromogenic LAL assay and a Water for Injection, USP Sterile Grade volume of 10 mL. Extraction solution volumes can vary depending on the kit application. The solution also functions as a fixed diluent. For example, for a product that requires an initial dilution of 1:10, the extraction solution volume will be 9.0 mL so a 1:10 dilution will be achieved when 1.0 mL of the sample is added to the solution contained in the vial. In addition, the extraction solution can also serve to prepare the sample for the LAL test with the addition of other ingredients, e.g. buffers, salts, etc., thus serving two functions simultaneously, i.e., dilution and sample treatment. The vial, being made of HDPE is also resistant to heat and has been shown not to significantly bind endotoxin or leach compounds that may interfere with the LAL assay. For example, some sample kits require dilution and heating of the sample under test prior to testing. The HDPE vials in the kits described in this invention can easily withstand temperatures of up to 100° C., either in a dry block or boiling water bath.

### Certification of Kits

The aforementioned procedures are applied in part or in total to each class of sample which in turn will define a ‘KIT’ (see examples below). Once an optimal LAL formulation, sample dilution, and/or sample treatment is determined, the KIT is ‘certified’ through replication employing standard endotoxin added to a representative sample containing undetectable endotoxin, or at a minimum, an endotoxin level sufficiently below the level of detection required for assessing the quality of the sample. Samples used for certification can be commercially available and found to have sufficiently low endotoxin or samples can be treated using a number of common procedures to lower or remove endotoxin. These samples are then spiked with standard endotoxin in various amounts and replicate assays run to determine the optimal conditions for acceptable results. In addition to the selection of a sample, certification also involves the determination of the requirement for dilution of the sample and what dilution vehicle (e.g. purified water, buffer, etc.) is optimal. In addition, for certain samples, both dilution and additional physical treatment may be required. One example of additional treatment is a heating step required to remove a biologically active component of the sample which would interfere with the assay (see EXAMPLE I). The certification of each type of kit therefore consists of a set of detailed instructions for performing the assay which if followed will result in an accurate determination of endotoxin in the sample within the range of detection specified for the kit.

### EXAMPLES OF TESTING KITS FOR VARIOUS PRODUCT APPLICATIONS

### Example I: Serum Testing Kit

The serum testing kit described in this invention consists of lyophilized LAL formulated for optimal reactivity with serum dilution/treatment packaged in capped tube 1, preferably plastic; HDPE vial containing 9.0 mL of Water for Injection, USP Sterile Grade 2; and polypropylene bulb pipets 3 and 4 calibrated to deliver 1.0 mL. The kit also includes written instructions 6 for carrying out the test and a certificate of analysis 7 memorializing the Limulus amebocyte lysate sensitivity, the nature and identity of the analyzed sample, the endotoxin free nature of the transfer instrument, the acceptable instrumentation needed for the analysis, and the incubation time and temperature required for the test. As will be noted in the certificate of analysis, the endotoxin content of raw, i.e., freshly collected bovine serum represent endotoxin from living and dead bacteria that can be used as an indicator of serum quality prior to batch pooling and sterilization.

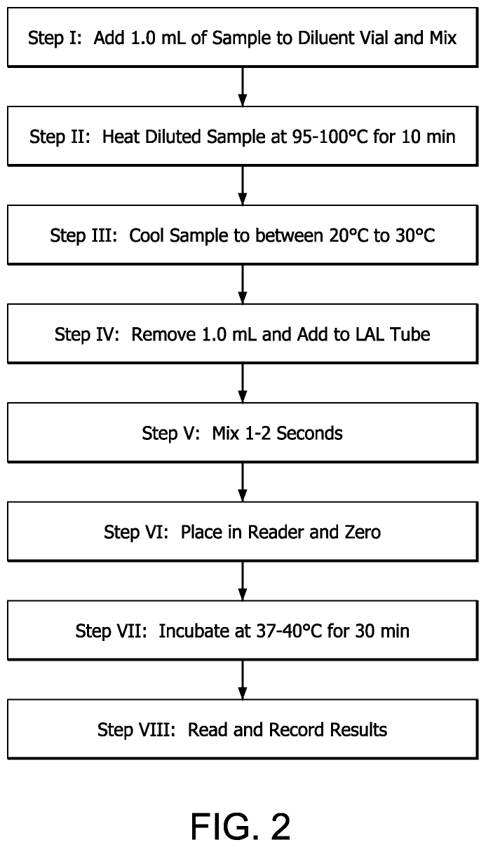

To use the kit of the present invention, 1.0 mL of a serum sample, e.g., freshly collected bovine serum collected, for example, in vial 5, is added via pipet 3 to HDPE vial 2, as identified in FIG. 2 as Step I. This results in a 1:10 dilution sufficient to prevent coagulation upon heating. HDPE vial 2 is then placed in a block heater set to 95-100° C. or in a boiling water bath for 10 minutes. Step II. Following heating, vial 2 is placed in an ice bath, freezer, or refrigerator until the contents cool to at least 37° C. but not less than 20° C. Step III. One (1.0) mL of this treated solution is removed using fresh pipet 4 and added to LAL tube 1. Step IV. Reconstitution is usually effected by gently tapping on tube 1 with a forefinger followed by rapid mixing using a laboratory mixer for a few seconds. Step V. Once the contents are thoroughly reconstituted, tube 1 is read in spectrophotometer or optical reader 8 equipped to accept a 12 mm diameter tube and possessing a light source with a wavelength of 380-660 nm (450 nm preferred). Step VI. The reading obtained should be 0.000 and represents the background or blank. Tube 1 is then placed in a block heater or biological incubator set at 37° C. for the period of time indicated in kit instructions 6. For serum testing, this period of time is 30 minutes. Step VII. Following incubation, tube 1 is again placed in spectrophotometer or optical reader 8, read and the reading is recorded. Step VIII. Instructions 6 which accompany the kit, contain specific values at or above which will indicate a certain concentration of endotoxin. The incubation period can also be changed to allow for the determination of either more or less endotoxin within the limits of the reagent/kit and accompanying instructions 6.

After the test is completed, certificate of analysis 7 can be completed with the relevant test information previously elucidated.

### Example II: Dialysate Testing Kit

The dialysate testing kit described in this invention consists of lyophilized LAL formulated for optimal reactivity with dialysate packaged in capped tube 1, preferably plastic; HDPE vial containing 9.0 mL of Water for Injection, USP Sterile Grade 2; and polypropylene bulb pipets 3 and 4 calibrated to deliver 1.0 mL. The kit also includes written instructions 6 and certificate of analysis 7, as previously described.

To use the kit of the present invention, as generically discussed with regard to Example I, 1.0 mL of a dialysate sample, e.g., USP dialysate, collected, for example in vial 5, is added via pipet 3 to HDPE vial 2. This results in a 1:10 dilution. One (1.0) mL of dilution is removed using fresh pipet 4 and added to LAL tube 1. Reconstitution is usually effected by gently tapping on tube 1 with a forefinger followed by rapid mixing using a laboratory mixer for a few seconds. Once the contents are thoroughly reconstituted, tube 1 is read in spectrophotometer or optical reader 8 equipped to accept a 12 mm diameter tube and possessing a light source with a wavelength of 380-660 nm (450 nm preferred). The reading obtained should be 0.000 and represents the background or blank. Tube 1 is then placed in a block heater or biological incubator set at 37° C. for the period of time indicated in kit instructions 6. Following incubation, tube 1 is again placed in spectrophotometer or optical reader 8, read and the reading recorded. Instructions 6 contain specific values at or above which will indicate a certain concentration of endotoxin. The incubation period can also be changed to allow for the determination of either more or less endotoxin within the limits of the reagent/kit and accompanying instructions 6.

After the test is completed, certificate of analysis 7 can be completed with the relevant test information previously elucidated.

### Example III: Water and Wastewater Testing Kit

The water and wastewater testing kit described in this invention consists of lyophilized LAL formulated for optimal reactivity with water or wastewater packaged in capped tubes 1, preferably plastic; HDPE vial containing 9.0 mL of Water for Injection, USP Sterile Grade 2; and polypropylene bulb pipets 3 and 4 calibrated to deliver 1.0 mL.

To use the kit of the present invention, as generically discussed with regard to Example I, 1.0 mL of a water or wastewater sample, collected, for example, in vial 5, is added directly to LAL tube 1 or is added via pipet 3 to HDPE vial 2. If a 1:10 dilution results, this is usually sufficient to lower the concentration in samples containing extremely high concentrations of endotoxin, resulting in a more accurate result. Reconstitution of tube 1 with the sample or sample dilution is usually effected by gently tapping on the tube with a forefinger followed by rapid mixing using a laboratory mixer for a few seconds. Once the contents are thoroughly reconstituted, tube 1 is read in spectrophotometer or optical reader 8 equipped to accept a 12 mm diameter tube and possessing a light source with a wavelength of 380-660 nm (450 nm preferred). The reading obtained should be 0.000 and represents the background or blank. Tube 1 is then placed in a block heater or biological incubator set at 37° C. for the period of time indicated in kit instructions 6. Following incubation, tube 1 is again placed in spectrophotometer or optical reader 8, read and the reading recorded. Instructions 6 contain specific values at or above which will indicate a certain concentration of endotoxin. The incubation period can also be changed to allow for the determination of either more or less endotoxin within the limits of the reagent/kit and accompanying instructions 6. As will be noted in the certificate of analysis, the endotoxin content of raw, i.e., freshly collected bovine serum represent endotoxin from living and dead bacteria that can be used as an indicator of the effectiveness of water treatment/purification equipment/systems.

After the test is completed, certificate of analysis 7 can be completed with the relevant test information previously elucidated.

### Example IV: Urine Testing Kit

The urine testing kit described in this invention consists of lyophilized LAL formulated for optimal reactivity with urine packaged in capped tube 1, preferably plastic; HDPE vial containing 9.0 mL of Water for Injection, USP Sterile Grade 2; and polypropylene bulb pipets 3 and 4 calibrated to deliver 1.0 mL.

To use the kit of the present invention, as generically discussed with regard to Example I, 1.0 mL of a urine sample collected, for example, in vial 5, is added via pipet 3 to HDPE vial 2. This results in a 1:10 dilution and removal of sample interference. Following dilution, 1.0 mL of the diluted sample is added to LAL tube 1. Reconstitution is usually effected by gently tapping on tube 1 with a forefinger followed by rapid mixing using a laboratory mixer for a few seconds. Once the contents are thoroughly reconstituted, tube 1 is read in spectrophotometer or optical reader 8 equipped to accept a 12 mm diameter tube and possessing a light source with a wavelength of 380-660 nm (450 nm preferred). The reading obtained should be 0.000 and represents the background or blank. Tube 1 is then placed in a block heater or biological incubator set at 37° C. for the period of time indicated in kit instructions 6. Following incubation, tube 1 is again placed in spectrophotometer or optical reader 8, read and the reading recorded. Instructions 6 contain specific values at or above which will indicate a certain concentration of endotoxin. The incubation period can also be changed to allow for the determination of either more or less endotoxin within the limits of the reagent/kit and accompanying instructions. As will be noted in the certificate of analysis, the endotoxin content of a urine sample suspected to represent a Gram-negative bacterial infection, if at or above a certain level as indicated in this assay, represents the presence of a certain level of bacteria shown by clinical studies to indicate an active urinary tract infection (UTI).

After the test is completed, certificate of analysis 7 can be completed with the relevant test information previously elucidated.

### Example V: Dairy Product Testing Kit

The dairy testing kit described in this invention consists of lyophilized LAL formulated for optimal reactivity with milk packaged in capped tube 1, preferably plastic; HDPE vial containing 10.0 mL of Water for Injection, USP Sterile Grade 2; and polypropylene bulb pipets 3 and 4 calibrated to deliver 0.04 mL per drop.

To use the kit of the present invention, as generically discussed with regard to Example I, one drop (0.04 mL) of a milk sample is added via pipet 3 to HDPE vial 2. This results in approximately a 1:250 dilution and removes sample interference (mainly opacity to allow for obtaining a blank reading in spectrophotometer or optical reader 8). Following dilution, 1.0 mL of the diluted sample is added to LAL tube 1. Reconstitution is usually effected by gently tapping on tube 1 with a forefinger followed by rapid mixing using a laboratory mixer for a few seconds. Once the contents are thoroughly reconstituted, tube 1 is read in spectrophotometer or optical reader 8 equipped to accept a 12 mm diameter tube and possessing a light source with a wavelength of 380-660 nm (450 nm preferred). The reading obtained should be 0.000 and represents the background or blank. Tube 1 is then placed in a block heater or biological incubator set at 37° C. for the period of time indicated in kit instructions 6. Following incubation, tube 1 is again placed in spectrophotometer or optical reader 8, read and the reading recorded. Instructions 6 contain specific values at or above which will indicate a certain concentration of endotoxin. The incubation period can also be changed to allow for the determination of either more or less endotoxin within the limits of the reagent/kit and accompanying instructions. As will be noted in the certificate of analysis, the endotoxin content of raw, i.e., unpasteurized or ultrahigh heat treated milk (UHT) represents endotoxin from both living and dead Gram-negative bacteria that can be used to assess the keeping quality or shelf life of the raw milk following treatment, especially UHT. The endotoxin level chosen for this kit reflects the levels shown by published research to affect milk quality.

After the test is completed, certificate of analysis 7 can be completed with the relevant test information previously elucidated.

Certain novel features and components of this invention are disclosed in detail in order to make the invention clear in at least one form thereof. However, it is to be clearly understood that the invention as disclosed is not necessarily limited to the exact form and details as disclosed, since it is apparent that various modifications and changes may be made without departing from the spirit of the invention.

1. A testing kit for the point of use detection of bacterial endotoxin in an aqueous solution sample using an optical measurement, said test kit comprising:
(a) at least one capped polystyrene tube containing freeze dried or air dried, endotoxin specific, horseshoe crab Limulus amebocyte lysate (LAL), whereby LAL sensitivity and kit performance relative to the sample is pre-certified to eliminate sample interference, including sample inhibition or enhancement, by the use of a fixed diluted sample;
(b) at least one disposable endotoxin-free transfer instrument; and
(c) at least one vial of diluent/extraction solution.

2. The testing kit of claim 1 wherein said horseshoe crab Limulus amebocyte lysate is from Limulus polyphemus.

3. The testing kit of claim 1 wherein the level of sensitivity of the testing kit for detecting endotoxin can vary based on the formulation of the Limulus amebocyte lysate, incubation time, and the dilution/extraction solution.

4. The testing kit of claim 2 wherein the amount of said Limulus amebocyte lysate is dried in a capped polyethylene tube.

5. The testing kit of claim 3 wherein said diluent/extraction solution is purified, distilled, sterile, non-sterile, or filtered water, water for injection, water for irrigation, reverse osmosis water, salt solution, or buffer.

6. The testing kit of claim 1 further comprising written instructions for carrying out the test.

7. The testing kit of claim 1 wherein a portable spectrophotometer or like optical reader is used to read test results, said spectrophotometer or optical reader being capable of reading at a wavelength of between 380 and 600 mm and accepting a 12 mm diameter glass or plastic tube.

8. The testing kit of claim 1 further comprising a written certificate of analysis of the Limulus amebocyte lysate sensitivity, the nature and identity of the analyzed samples, the endotoxin-free nature of the transfer instrument, the acceptable instrumentation needed for the analysis, and the incubation time/temperature required for the test.

9. The method of detecting and measuring bacterial endotoxin in an aqueous solution sample using optical measurement, the steps of the method comprising:
obtaining an aqueous solution sample;
providing a liquid diluent solution;
adding the aqueous solution sample to the diluent solution;
mixing the aqueous solution sample and diluent solution to form a diluted sample;
heating the diluted sample;
providing a freeze dried or air dried endotoxic specific, horseshoe crab Limulus amebocyte lysate which is pre-certified to eliminate sample interference;
adding the diluted sample to the pre-certified Limulus amebocyte lysate;
mixing the diluted sample to the pre-certified Limulus amebocyte lysate;
incubating the mixed diluted sample and pre-certified Limulus amebocyte lysate;
providing a spectrophotometer or like optical reader;
positioning the mixed diluted sample and the pre-certified Limulus amebocyte lysate in the spectrophotometer or optical reader; and
determining the presence and concentration of endotoxin in the aqueous solution sample by reading the resulting values shown by the spectrophotometer or optical reader.

10. The method as in claim 9 wherein the amebocyte lysate is from Limulus polyphemus.

11. The method as in claim 9 wherein the liquid diluent solution is purified, distilled, sterile, non-sterile, or filtered water, water for injection, water for irrigation, reverse osmosis water, salt solution, or buffer.
